(19) United States
(12) Patent Application Publication
(10) Pub. No.: US 2001/0048923 A1
(43) Pub. Date: Dec. 6, 2001

(54) IMMUNE CELL REGULATION WITH SRC PTK SEQUESTERING AGENTS

(76) Inventors: JANET M. OLIVER, ALBUQUERQUE, NM (US); BRIDGET S. WILSON, ALBUQUERQUE, NM (US); ENRIQUE ORTEGA, MEXICO CITY (MX)

Correspondence Address: PEACOCK MYERS AND ADAMS P C, P O BOX 26927, ALBUQUERQUE, NM, 871256927

(21) Appl. No.: 09/052,929

(22) Filed: Apr. 1, 1998

(51) Int. Cl. A61K 39/395

(52) U.S. Cl. 424/143.1

(57) ABSTRACT

Signal propagation in MIRR-coupled immune cell signalling pathways is extinguished or curtailed by sequestering Src family enzymes indigenous to the cell. Signal-curtailing ligands which cross-link MIRR extracellular domains and stabilize intra-cellular Src/MIRR complexes are exemplary sequestering agents which function as immune cell antagonists.

The research leading to the inventions described in this patent application was partially funded by NIH Grant #RO1 GM 49814 and R03 TW00440, and the U.S. Government has certain rights in any patent issuing on this application.

### BACKGROUND OF THE INVENTION

1. Field of Art

Cells of the immune system express multichain immune recognition receptors (MIRRS) which initiate cell effector function when immunostimulated. It was long assumed that resting cells of the immune system were activated by simple binding of these receptors with a sufficient number of appropriate ligands.

Quite recently, however, it has been shown that immune cell activation is far more complicated, and that cell responses to MIRR ligation can vary qualitatively as well as quantitatively depending upon the ensuing transmembrane events which initiate cell responses and the cytoplasmic events which propagate these responses. Characterization of these events, collectively referred to herein as “MIRR signalling pathways” has recently been the subject of much research, as such knowledge should enable the development of agonists, partial agonists, and antagonists for therapeutic regulation of immune cell function. Of particular clinical interest are partial agonists and antagonists for inhibiting immune cell activation (negative or “down” regulation) in patients with immune cell mediated autoimmune diseases, asthma, and allergic disorders.

2. Discussion of Related Art

One of the first events in the MIRR signalling pathway leading to activation of immune cell effector functions such as cellular cytotoxicity, production of IL-2, and basophil degranulation is the phosphorylation of tyrosine residues in a conserved peptide “motif” present in the signalling subunits of the receptor. The motif, termed ITAM (immunoreceptor tyrosine-based activation motif) has been reported to be present in each of the signalling subunits of T-cell receptors (TCRs), B-cell antigen receptors (BCRs), high affinity IgE receptors (FcεRIs), and most IgG receptors (FcyR1, FcyRIIA, FcyRIIC and FcyRIII), and is probably ubiquitous in immune cells. ITAM phosphorylation is mediated by one or more protein tyrosine kinases (PTKs) of the Src family; the phosphorylated ITAMs in turn bind one or more PTKs of the Syk family (e.g., Syk and Zap-70), resulting in Syk PTK activation (Chan & Shaw, “Regulation of antigen receptor signal transduction by protein tyrosine kinases”, Current Opinion in Immunol. 8:394-401,1995; Cambier, J. Immunology 155:3281-3285, 1995; both incorporated herein by reference). Activated Syk in turn recruits and activates down stream effectors for physiological responses in the cell.

This model of MIRR signal transduction recognizes the role played by sequential activation of the Src- and Syk-families of tyrosine kinases in the phosphorylation of ITAM to mediate downstream intracellular effector functions of immune cells. However, Chan and Shaw frankly state that their review of studies leading to this model do not elucidate the mechanisms of the phosphorylation cascade mediated by ITAMs. In particular, the review states that the physical basis for the binding of specific members of the Src family of PTKs such as Fyn and Lyn to unphosphorylated ITAMs “remains unclear”. It is also unclear how receptor ligation activates these Src PTKs, and how they recognize ITAMs as their main or only targets for phosphorylation.

It has been postulated for several years that aggregation or clustering of immune cell surface receptors with each other or with receptor-associated proteins is a prerequisite for initiation of receptor signal transduction and activation of resting immune cells (J. Immunol. 155:3281, 1995). Chan and Shaw (op.cit., p. 394) describe the clustering or aggregation of MIRRs required for signal initiation as dimerization, oligomerization, or cross-linking of immune cell receptor subunits. Ortega et al. (EMBO J., 7:4101-4109, 4101, 1988) discuss the effect of orientational constraints as well as the extent of cross-linking on the binding and magnitude of secretory responses induced by ligating the FcεR1 of mast cells. Science, op.cit. p.459, describes differences in both qualitative and quantitative responses of T-cells induced when slightly altered antigenic peptides bind their receptors. Ligands of choice for such studies have included monoclonal antibodies (mAbs) to cell membrane receptors as model agonists, which mimic cellular activation by natural antigen agonists. The mAbs selected are typically homogeneous and structurally defined cross-linking agents which permit, for example, rigorous quantitative analysis of the relationships between receptor aggregation and secretion (EMBO J., op.cit. p. 4101; Science 267: 515-518, 1995).

While these studies have provided basic insights into immune cell activation, mechanisms which would permit clinical regulation of immune cells by altered cellular responses to MIRR ligation have not been identified or characterized. In particular, no consistent differences between ligands have been heretofore characterized in kinetic studies of association or dissociation from receptors, or in biophysical studies of cross-linked receptors, including properties of lateral and rotational mobility, conformational differences, and binding lifetime, which would permit the identification of ligands for clinical use having predictable effects on a broad spectrum of immune cell responses.

### BRIEF DESCRIPTION OF THE DRAWING

FIG. 1: Ins(1,4,5)P₃ synthesis and CA²⁺ mobilization induced by FcεR1 dimers and oligomers

In A, RBL-2H3 cells were incubated for various times with one μg/ml DNP-BSA (igE-primed cells) or with 70 nM mAbs (unprimed cells). Reactions were stopped with TCA and levels of Ins(1,4,5)P₃ in the resulting supernatants were measured. Each point is an average from two separate experiments, each performed in duplicate. In B-D, cytoplasmic CA²⁺ levels were measured in individual Fura-2-loaded cells by ratio imaging microscopy. Antigen (100 ng/ml DNP-BSA) or mAbs (70 nM J17 or H10) were added at the time points indicated. Results show the CA²⁺ responses of 3 or 4 cells per experiment.

FIG. 2: Effects of FcεR1 crosslinking on anti-phosphotyrosine immune complex kinase activities.

RBL-2H3 cells were incubated for 5 min at 37° C. with no addition (column 1), 1 μg/ml DNP-BSA (column 2) or with 70 nM mAbs (columns 3,4,5). Cells were lysed and tyrosine phosphorylated proteins precipitated from lysates by incubation with Mab PY20 precoupled to protein A/G beads. The immune complexes were incubated for 5 min with [³²P]-ATP and the resulting phosphoproteins were separated by SDS-PAGE and detected by autoradiography. The migration of mol. wt. markers is indicated and known proteins among the principal phosphorylated bands are identified. H10-receptor dimers induce maximum in vitro phosphorylation of Lyn and receptor subunits.

FIGS. 3 and 4: Three phospho-β isoforms are induced by FcεR1 crosslinking but only two associate with Lyn.

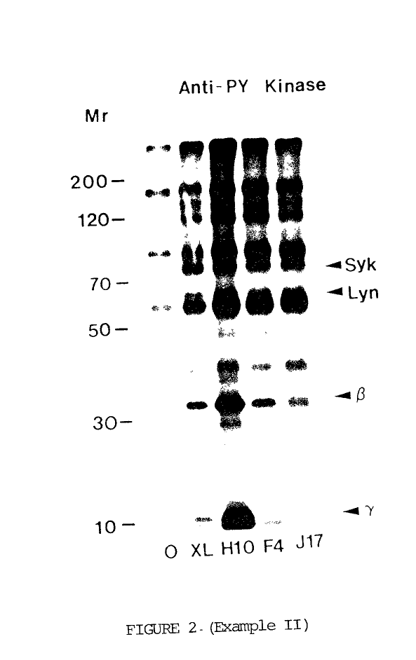

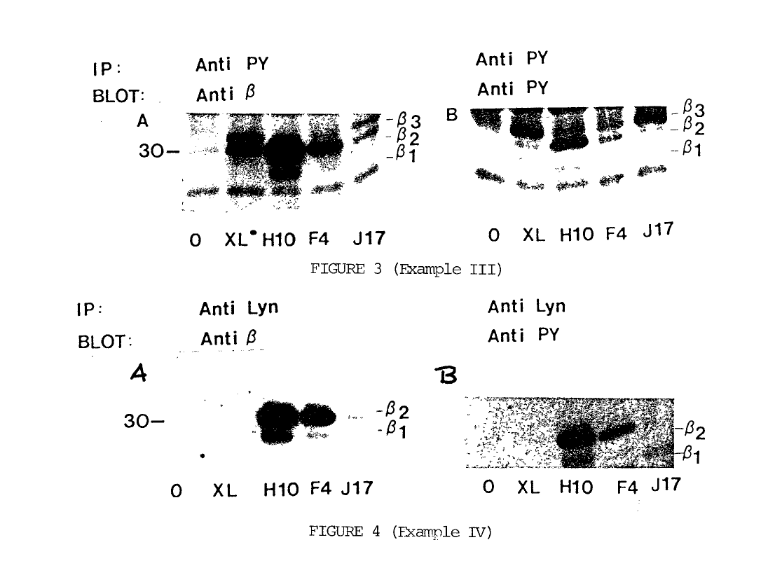

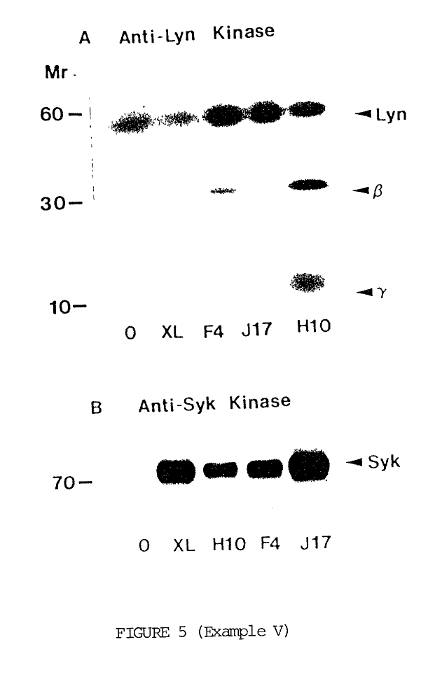

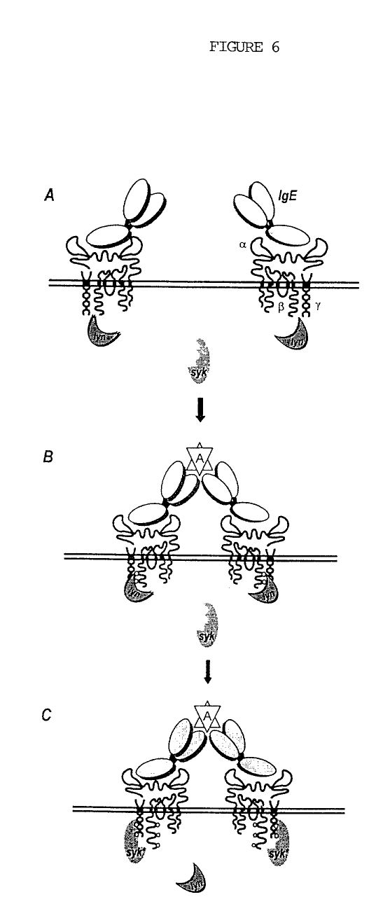

Cells were activated for 5 min with antigen or with mAbs and anti-PY20 (FIG. 3) or anti-Lyn (FIG. 4) immune complexes were generated as described for FIG. 2. Proteins were separated on duplicate 10% SDS gels and transferred to nitrocellulose for Western blotting using anti-β (3A, 4A) or anti-PY20 (3B, 4B) mAbs followed by [¹²³I]-labelled donkey anti-mouse IgG. After blotting, the gels were dried and radiolabelled proteins detected by autoradiography. Results of anti-β blotting (A) show clearly that activated RBL-2H3 cells contain three distinct phospho-β isoforms. Results of anti-phosphotyrosine blotting (B) show that the three isoforms represent a phosphorylation series. Results in FIG. 4 establish that Lyn associates only with the less phosphorylated β1 and H10-induced β2 isoforms.

FIG. 5: Effect of FcεR1 crosslinking on anti-Lyn and anti-Syk immune complex kinase activities.

The experiment was as described in FIG. 2 except that all crosslinking agents were added for 5 minutes at 37° C. and immunoprecipitation was with anti-Lyn (A) or anti-Syk (B). Anti-Lyn immune complexes (A) from resting, antigen-activated and mAb-activated cells could all autophosphorylate Lyn in vitro. Substantial amounts of FcεR1 β and γ subunits co-precipitated with Lyn from lysates of mAb H10-treated cells and were detected by their in vitro phosphorylation (lane 5). Anti-Syk immune complexes (B) from activated (lanes 2-5), but not resting (lane 1) cells, phosphorylated Syk in vitro. Syk activation was greatest in antigen and J17-treated cells and least in H10-treated cells.

FIG. 6: Model of MIRR signalling cascade in high-affinity IgE receptors.

Panel A: Resting immune cell. Src PTK (Lyn) associated with FcεR1 signalling subunit cytoplasmic tails. Syk PTK floats free.

Panel B: Cross-linking of IgE receptors. Lyn binds to subunits and mediates transphosphorylation. Syk still floats.

Panel C: Signal propagation. Lyn dissociates from phosphorylated subunits permitting Syk recruitment to subunit phospho-ITAMs.

### SUMMARY OF THE DISCLOSURE

The present inventions are predicated on the discovery of a heretofore unrecognized regulation step in immune cell signalling pathways which are characterized by Src PTK-mediated MIRR signalling subunit ITAM phosphorylation in response to MIRR immunostimulation, followed by Syk tyrosine kinase activation. According to the present discovery, propagation of signals initiated by ITAM phosphorylation along this pathway to the cell interior is dependent upon dissociation of Src PTK from its phosphotyrosine binding sites on the receptor after subunit phosphorylation has gone to completion. If this event fails to occur, activated Src remains in a complex with the receptor subunits, Syk PTK and/or ZAP-70 are not activated, and signal transmission along this pathway is substantially curtailed or extinguished. Since activation of Syk kinase is a critical downstream step for the eventual activation of effector functions of resting immune cells, these effector functions can be blocked by inhibiting dissociation of these Src PTK/phosphotyrosine complexes employing the Src PTK sequestering agents of the invention.

The invention accordingly provides a method for selectively inhibiting immune cell effector functions by blocking multichain immunoregulatory cell surface receptors (MIRRs) that normally respond to signalling-competent ligands by activation of Src family PTKs to mediate tyrosine phosphorylation of MIRR signalling subunits, followed by activation of Syk family PTKs. Blocking is accomplished by activating and sequestering the Src family member(s) peculiar to the cell as Src-receptor complexes.

The invention further provides immune cell blocking agents or antagonists comprising Src sequestering agents which stabilize Src PTK/phosphotyrosine complexes to block Syk activation and inhibit immune cell activation via this pathway. In one embodiment, the antagonists are ligands that both activate Src PTKs to initiate MIRR subunit tyrosine phosphorylation and also stabilize the complexes to block Syk activation and immune cell activation.

The inventions are broadly useful for treating immune disorders and diseases characterized by inappropriate immune cell responses, for example, for treating allergies and asthma by suppressing mast cell and basophil activation, and for treating autoimmune diseases and for inhibiting transplant rejection by suppressing T-cell and B-cell activation.

The inventions are also useful in understanding and treating infectious diseases. Specifically, some pathogens may elude immune killing by producing peptide antigens that inhibit desirable T-cell activation by this pathway. Recognizing and eliminating such antigens may provide new approaches to chronic infections. Additionally, their recognition and elimination may improve the effectiveness of vaccines intended to confer immunity against such pathogens.

### DETAILED DESCRIPTION OF THE INVENTION

Multichain immune recognition receptors (MIRRs) expressed by immune system cells comprise repeating immunoglobulin loops reactive with activating (signal competent) ligands in their extracellular domains, and immunoreceptor tyrosine-based activation motifs (ITAMs) in their cytoplasmic tails that induce cellular effector responses. Known MIRR family members include the high affinity IgE receptor, FcεR1, of mast cells and basophils; the T cell receptor, TCR; the B cell receptor, BCR; and membrane IgG receptors, including FcγR1, FcyRII and FcyRIII isoforms of myeloid cells. Members of the MIRR family are responsible for many diverse immune cell functions such as histamine release in basophils and mast cells; cytokine production in T-cells, basophils, eosinophils, mast cells and many myeloid cells; antibody production in B-cells; tumor cell killing in NK cells and T- cells; and host response to pathogens in monocytes, macrophages, and neutrophils. Activating the IgE receptor FcεR1 initiates allergic and asthmatic diseases. Inappropriate activation of other MIRRs is implicated in autoimmune diseases and transplant rejection.

In the normal course of events in vivo, ligating (cross-linking) MIRR extracellular receptor subunits with signalling-competent ligands such as natural antigens or anti-receptor antibodies activates one or more members of the Src family of tyrosine kinases and initiates the Src-mediated phosphorylation of tyrosines on the ITAMs located within the cytoplasmic tails of one or more of the receptors' subunits. Activated Src kinases bind to these phosphotyrosines, enhancing their access to more ITAM tyrosines and creating substantially fully phosphorylated ITAM binding sites for other proteins, including members of the Syk tyrosine kinase family, as Src PTKs dissociate. Subsequently, Syk kinases are recruited to specific phosphorylated ITAM binding sites to form Syk-phospho-ITAM complexes which result in Syk autophosphorylation and activation (FIG. 6). Activated Syk then phosphorylates signalling proteins that initiate activation of one or more pathways for cell effector function response. For example, in primary and cultured basophils and mast cells, crosslinking the high affinity IgE receptor, FcεR1, activates a signalling sequence that leads within minutes to degranulation and membrane/cytoskeletal responses, including actin polymerization, ruffling, spreading, integrin activation and actin plaque assembly, and within hours to increased cytokine synthesis. Src and Syk PTKs are known in the art; also as known in the art, specific immune cell types harbor specific kinases.

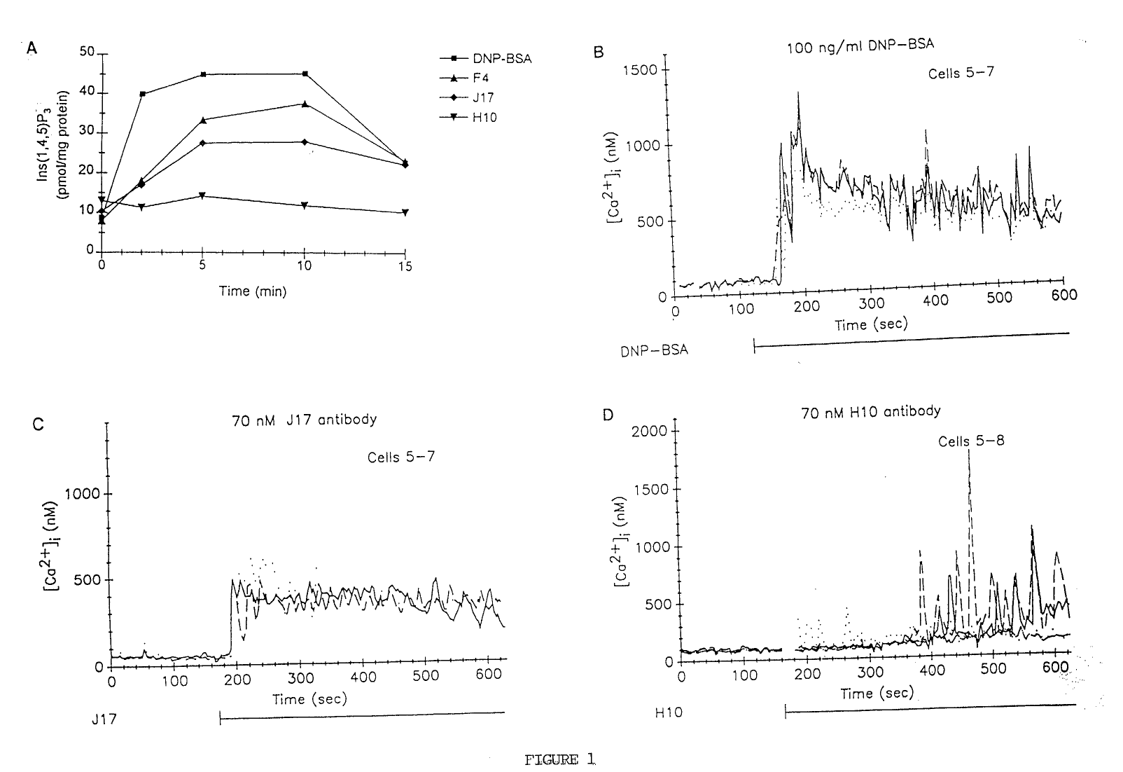

Some early events by which cross-linking this multichain receptor leads to functional responses have been described. RBL-2H3 rat tumor mast cells, for example, contain two receptor-associated PTKs, the Src-family enzyme, Lyn, whose principal substrates are the receptor's β and γ subunits, and the Syk family parent enzyme Syk/PTK72 that phosphorylates a wide range of downstream signalling molecules including PLCγ isoforms, the p85 subunit of phosphatidylinositol 3-kinase, Vav, Grb2 and others. In resting RBL-2H3 cells, a proportion of Lyn normally loosely associates (see, e.g., FIG. 1) with the FcεR1 β and γ subunit; FcεR1 crosslinking with competent ligand activates this subpopulation of Lyn, resulting in the phosphorylation of tyrosines within the ITAMs found in both the FcεR1 β and γ subunits (J.Biol.Chem. 2:11185-11192,1997) and the creation of phosphotyrosine binding sites for the recruitment and activation of more Lyn molecules (Proc.Nat.Acad.Sci.USA 91:11251-11255,1994).These phosphotyrosines also bind other signalling molecules containing SH2 domain motifs. In particular, doubly phosphorylated FcεR1 γ subunit ITAMs serve as binding sites for the tandem SH2 domains of Syk, resulting in its autophosphorylation and activation (J.Biol.Chem. 270:10498-10592, 1995). This model of signal initiation by sequential kinase activation is characteristic of MIRR signalling cascades in immune system cells. ITAM motifs are prevalent in the cytoplasmic tails of other multichain immune cell receptors and engagement of these receptors with ligand activates these cells through a corresponding sequential activation of receptor-associated Src and Syk kinases.

The important early biophysical events of this natural process for activation of immune system cells include, sequentially, 1) binding of the extracellular domain of a MIRR receptor to a ligand, creating a ligand-receptor complex competent to activate one or more Src PTKs and initiate Src-mediated phosphorylation of ITAM tyrosine residues; 2) binding of the activated Src PTKs to phosphotyrosine binding sites on the partially phosphorylated receptor to form a MIRR/Src PTK complex sufficiently stable to block Syk PTK binding to the receptor and Syk activation; and 3) dissociation of Src from the fully phosphorylated MIRR to allow Syk recruitment and signal propagation.

According to the invention, normal in vivo immune cell activation is down-regulated by disrupting this process between events 2) and 3), i.e., by inhibiting full MIRR phosphorylation that in turn disallows Src dissociation and subsequent Syk recruitment to the receptor tails, a crucial prerequisite for downstream activation of Syk-dependent cell effector functions. This discovery permits the clinical intervention in the arousal of resting immune cells in response to activators such as allergens by sequestering Src family enzymes, thus curtailing or extinguishing signal propagation along this pathway.

In a preferred embodiment of the invention, this is accomplished by cross-linking extracellular subunits of the MIRR receptors with a multivalent, preferably divalent, ligand to form multimeric, preferably dimeric, cross-linked subunits which are effective to sequester the Src PTK(s) specific to the immune cells of interest. Ligands suitable for this practice of the invention comprise those ligands capable of signalling the induction of the Src PTK-mediated phosphorylation cascade in MIRR-coupled signalling pathways of immune cells without significant downstream activation of SykPTK. In particular, these ligands are not sufficiently signal-competent to effect Src PTK-mediated phosphorylation of the MIRR ITAMs to the degree necessary to promote the dissociation of the MIRR/Src PTK complexes which must precede SykPTK activation and induce downstream SykPTK-dependent cell effector functions. These undissociated Src/receptor complexes are herein referred to as “stable MIRR/Src PTK complexes” or “stable complexes” or “Src-sequestering complexes”. Agents, either extracellular or intracellular, which promote the sequestration of Src PTK to inhibit Syk activation are referred to herein as “Src sequestering agents” or “Src sequesterers”. Ligands which cross-link the extracellular domains of the MIRRs and promote the formation of Src-sequestering complexes are herein referred to as “signal-curtailing ligands”. ITAMs insufficiently phosphorylated to promote Src PTK dissociation are referred to herein as “incompletely phosphorylated ITAMs”.

The ligands of the invention are partial agonists or antagonists of immune cells having MIRR-coupled signalling pathways which normally include an Src PTK-mediated phosphorylation cascade with sequential Src PTK and Syk activation. Since all MIRRs activate cells by essentially the same pathway, ligands or other Src sequestering agents which block signal progression along this pathway by stabilizing complexes between activated Src and incompletely phosphorylated ITAMs are effective for blocking MIRR-activated effector functions dependent upon SykPTK activation in all immune cells having MIRRs. Owing to the early blockage of the MIRR-coupled signalling pathway according to the invention, many, if not most, immune cell effector functions can be regulated by selection of appropriate ligands for particular MIRRs. This result is possible because, unexpectedly, the Src PTKs involved in this mechanism are “rate-limiting”: i.e., there can be more activated receptors than there are Src PTK molecules available to phosphorylate them. Src activation does not increase in response to a decrease in cell concentration of the free active enzyme, and locking the enzyme to one phosphotyrosine binding site leaves another binding site bereft of its needed signalling molecule.

The key physiologic properties of useful ligands are (1) no substantial or significant independent signalling activities and (2) their ability to inhibit signalling induced by normally signalling-competent MIRR ligands; and (3) their ability to successfully compete with normally signalling-competent ligands to form stable complexes with the MIRR extracellular receptors. The key biochemical properties of useful ligands are (1) the ability to activate Src tyrosine kinases to phosphorylate MIRR signalling subunits; (2) the ability to sequester cell Src PTKs by inducing the formation of stable complexes between the activated Src kinases and their phosphotyrosine binding sites on incompletely phosphorylated receptor units of the MIRRs of interest, and (3) their inability to substantially or significantly activate Syk kinases. Exemplary ligands comprise oligopeptides with these properties, especially naturally-occurring receptor ligands and anti-receptor antibodies, either mono- or polyclonal. Any ligands that meet the criteria set forth herein can be used, including synthetic ligands. For long-lasting cell inhibition, protease-resistant ligands are preferred.

Guidelines for selecting ligands useful in the practice of the invention are set forth infra. Briefly, it is recommended that secretion and binding screening assays be first performed to evaluate the competitive binding affinity of the ligand for the cell receptor, and its effect on cell secretory functions, as these assays are relatively simple to perform and will eliminate useless ligands. Exemplary assays employing monoclonal antibodies as ligands are described in EMBO J. 7:4101-4109, Ortega, et al., 1988, incorporated herein by reference. After screening, poor secretogogues with high receptor affinity are evaluated for Src/MIRR subunit complexes, typically by immunoprecipitation from cell lysates using anti-receptor or anti-Src antibody, followed by SDS-PAGE separation (Examples). Cross-link-induced Syk activation is then measured (Examples). Screened ligands which both produce stable complexes of Src PTK and MIRR subunits and are poor Syk activators are selected for in vivo use according to the invention. An exemplary ligand useful in the practice of the invention, mAb H10, is described in the Examples and in the prior art (EMBO J., op.cit.).

The above-described ligands are preferred as Src sequestering agents, inter alia, because of the relative ease with which they can be clinically used. Since their targets are the extracellular domains of the MIRRs, transmembrane carriers are not necessary. Also, the preferred ligands are readily soluble in water or water-miscible solvents, and are conveniently administered in the form of aqueous pharmaceutical compositions. Physiological saline-based vaccines for intramuscular (slow effect) or intravenous (rapid effect) administration are exemplary, as are aerosols for inhalation containing aqueous solutions of the ligands and conventional pharmaceutical propellant. Topical application of pharmaceutical compositions of the ligands, optionally in a carrier which promotes skin penetration, is also contemplated. Suitable carriers and adjuvants for the desired applications are well-known in the art and are readily selected for the disease or disorder to be treated. However, intracellular or other extracellular Src PTK sequestering agents can also be used in the practice of the invention as they are developed, with the proviso that these agents meet the criteria described herein, and as set forth in the claims.

The following Examples describe simple in vitro tests for selecting ligands for use in the practice of the invention.

1) Activation of Src Kinase and Src-Receptor Complex Formation

Examples II and IV: Anti-Lyn (or other Src family member) or anti-MIRR subunit immunoprecipitation, followed by in vitro kinase assay (FIG. 5A).

2) Impaired Activation of Syk Kinases

Example VI: Anti-Syk immune complex assay (FIG. 5B)

### EXAMPLES

### Materials and Methods

Reagents:

The preparation and characterization of three anti-FcεR1 mAbs, H10 (IgG2b), F4 (IgG1) and J17 (IgG1) is described in EMBO J., op. cit. Polyclonal anti-PY antibody was produced by G. Deanin and J. Potter, UNM and purified as described in Oncogene 2: 305-315, 1988. Monoclonal anti-IgE antibody (J Immunol. 124: 2728-2737, 1980) was purified from ascites. Rabbit anti-IgE antibody was prepared as in J. Cell Physiol. 148: 139-151, 1991. Mab to the FcεR1 β subunit was a generous gift of Dr. J. Rivera, NIH, U.S. Department of Health and Human Services. Rabbit anti-Syk antibody raised against a Syk-specific peptide was a generous gift from R Geahlen, Purdue University. Mouse monoclonal anti-phosphotyrosine antibody, PY20, and rabbit polyclonal anti-Lyn antibody were purchased respectively from Santa Cruz (Calif.) and Transduction Laboratories, Lexington, Ky.

Cell Activation:

For studies of the signalling activity of FcεR1 oligomers, cells were incubated overnight with 1 μg/ml anti-DNP-IgE, then washed with modified Hanks' buffer (J.Exp.Med. 135: 376-387, 1972) containing 0.1% bovine serum albumin (Hanks'-BSA) and activated by the addition of either 0.1 or 1.0 μg/ml of DNP-BSA (Molecular Probes, Eugene, OR, USA) or 1 μg/ml rabbit anti-IgE. For studies of the signalling activities of anti-FcεR1 mAbs, cells were simply suspended in Hanks'-BSA medium and activated by the addition of Mab.

Secretion:

Secretion was measured from the release of preloaded [³H]-serotonin (DuPont NEN) from cells grown as monolayers on 24-well tissue culture dishes as described in J.Biol.Chem. 270: 4013-4022, 1995.

Ins(1,4,5)IP₃ Levels:

Ins(1,4,5)IP₃ levels were determined in TCA precipitates of activated cell suspensions using the isomer-specific radio receptor assay of Challis et al., Biochem. Biophys.Res.Comm. 157: 684-691, 1988, as modified by Deanin et al., Biochem.Biophys.Res.Comm. 179: 551-557, 1991.

Ca²⁺ Mobilization:

[Ca²⁺]I was measured in individual, fura-2/AM-loaded RBL-2H3 cells using fluorescence ratio imaging microscopy as described in Mol.Biol.Cell 6: 825-839, 1995.

Microscopy:

To observe crosslinker-induced membrane ruffling and spreading, cell monolayers on glass coverslips were activated, then fixed either with 2% glutaraldehyde for scanning electron microscopy (as in J.Cell.Biol. 101: 2145-2155, 1985) or with 2% paraformaldehyde/x% Saponin followed by rhodamine-phalloidin (as in J.Immunol. 152: 270-279, 1994). Cells were examined using a Hitachi S800 SEM or a Zeiss Photomicroscope II equipped for epifluorescence microscopy.

Immune Complex Phosphatase Assays:

Cell suspension (6×10⁶ cells/ml; 1 ml per assay) were activated with antigen or Mab, then lysed in 50 mM Hepes, pH 7.2, 150 mM NaCl, 1% Brij-96 plus 1 μg/ml each of leupeptin, antipain and pepstatin. The clarified lysates were incubated for 2 hours at 4° C. with anti-phosphotyrosine antibody (PY20) pre-bound to protein A/G-Sepharose beads. Immune complexes were collected by centrifugation, washed 4 times in lysis buffer and incubated at 37° C. for 12h in 200 μl of the phosphatase buffer described by Pani et al. (1995) (62 mM Hepes pH 5.0, 6.25 mM EDTA, 12.5 mM dithiothreitol, 4 mM ρ-nitrophenyl phosphate.) Reactions were terminated by addition of 0.8 ml of 200 mM NaOH and absorbtion was measured at 410 nm using a Beckman spectrophotometer.

Immune Complex Kinase Assays:

Cells were activated and lysed as described above. In most experiments the clarified lysates were precleared of any detergent-soluble Mab-FcεR1 α subunit complexes by incubation for 2h at 4° C. with protein A- or Protein A/G-Sepharose beads. After preclearing (omitted when immunoprecipitation was with directly bead-coupled antibodies), they were incubated for 2h at 4° C. with specific antibodies pre-bound to Protein A-Sepharose (polyclonal anti-Lyn and anti-Syk antibodies), Protein A/G-Sepharose (monoclonal anti-phosphotyrosine antibodies) or with anti-phosphotyrosine-agarose or anti-Lyn agarose beads. After washing 4 times, kinase activity was determined from the incorporation of [γ-³²P]-ATP into specific proteins during a 2 min incubation at 30° C. with 10μCi[³²P]-ATP as described in Wilson et al (1995).

Immunoblotting:

Cells were activated, lysed, precleared (if appropriate) and specific proteins immunoprecipitated with antibody as above. Antibody-protein complexes were released from the washed beads by boiling, separated by 10% SDS-PAGE and transferred to nitrocellulose. After overnight incubation at 4° C. in 3% BSA to block non-specific binding, blots were probed with specific antibody for 1h at room temperature and washed again. For autoradiography, blots were incubated for a further hour with ¹²⁵I-labelled anti-mouse IgG and dried membranes were exposed to X-ray film. For chemiluminescence detection, blots were incubated with horse radish peroxidase (HRP)-conjugated anti-mouse antibodies, washed, immersed in ECL chemiluminescence solution and exposed to X-ray film.

### Example I

Different mAbs Induce Different Signalling Responses.

A. Secretion:

The mAbs used here were originally selected for their ability to compete with each other and with IgE for binding to the FcεR1 α subunit and to elicit different secretory responses (EMBO J. op. cit.). Originally, 10 nM antibody elicited optimal secretion and the order of potency was antigen=F4>J17>>H10. With current Mab preparations and RBL-2H3 cells, 70 nM concentrations of all three mAbs are necessary to induce optimal secretion and the order of potency has changed somewhat. Mab H10 remains a poor secretagogue, consistently inducing release of 15-20% of total [3H]-serotonin under conditions where optimal antigen (0.1 or 1.0 μg/ml DNP-BSA) causes the release of 40-70% of total mediator, depending on cell culture density and passage number. Current preparations of Mab J17 are strong secretagogues, inducing only 5-10% less secretion than antigen in a 20 min assay. Mab F4-receptor dimers have been less consistent, originally inducing more secretion than Mab J17 (EMBO J. op.cit.) but recently inducing responses that are smaller than J17-induced responses. Based on all assays of degranulation, the order of potency in the RBL-2H3 cells used here is: multivalent antigen >or =J17> or =F4>>H10.

B. Synthesis of Ins(1,4,5)IP₃:

The activation of PLCY isoforms, leading to the synthesis of Ins(1,4,5)IP₃, is one of the earliest responses of RBL-2H3 cells to FcεR1 crosslinking. The results in FIG. 1 show that multivalent antigen and mAbs F4 and J17 all induce an increase in cytoplasmic Ins(1,4,5)IP₃ levels that is detectable after 1 min and persists for around 10 min before returning towards baseline. In contrast, Mab H10 induces very little Ins(1,4,5)P₃ synthesis in RBL-2H3 cells.

C. Ca²⁺Mobilization:

Secretion depends on Ca²⁺ mobilization that is initiated by the _{Ins}(1,4,5)P₃-mediated release of intracellular stores and maintained by Ca²⁺ influx. We used ratio imaging microscopy to compare the Ca²⁺ mobilization responses of RBL-2H3 cells to Mab-induced receptor dimers and to DNP-BSA-induced receptor oligomers. Typical results are shown in FIG. 1. In duplicate experiments, 100% of cells showed Ca²⁺ responses to 1 μg/ml DNP-BSA (FIG. 1B) and to 70 nM mAbs J17 and H10 (FIGS. 1C, 1D). However, there were characteristic differences in the lag time to the initial Ca²⁺ spike response, previously attributed to the release of Ca²⁺ from intracellular stores: 42 sec for DNP-BSA-stimulated cells; 70 sec for J17-stimulated cells; and 170 sec for H10-stimulated cells. Additionally, antigen and Mab J17 induced a persistent increase in cytoplasmic Ca²⁺ levels, attributable to Ca²⁺ influx (Mol.Biol.Cell, op.cit.), whereas Mab H10 induced a series of Ca²⁺ spikes with little elevation in baseline Ca²⁺ levels. These data suggest that H10-induced receptor dimers can support the periodic release and re-uptake of Ca²⁺ stores. Nevertheless, these dimers induce very little Ca²⁺ influx. In a single experiment, Mab F4-receptor complexes induced responses that were similar to J17 (not shown).

D. Membrane and Cytoskeletal Responses:

FcεR1 crosslinking with multivalent antigen induces striking membrane and cytoskeletal responses including F-actin polymerization, membrane ruffling, spreading, integrin up regulation and the assembly of specialized adhesion structures called actin plaques. A subset of these responses was examined, including spreading, actin plaque assembly and membrane ruffling, by fluorescence microscopy of cell monolayers labeled with rhodamine-phalloidin to localize F-actin and by scanning electron microscopy. Resting cells adhere loosely to glass coverslips and distribute F-actin as a cortical meshwork that outlines pseudopodia as well as in amorphous cytoplasmic aggregates. These cells maintain a microvillous surface morphology. Crosslinking with DNP-BSA for 10 min induces a strong spreading response, accompanied by the assembly of actin plaques at sites of cell-substrate interaction and by a transformation of the upper cell surface to a lamellar topography. Crosslinking with mAbs F4 and J17 induces spreading, actin plaque assembly and ruffling responses that are essentially the same as those induced by antigen. In contrast, H10-activated cells show a modest spreading response, with little or no actin plaque assembly or membrane ruffling.

### Example II

Different mAbs Induce Different Phosphoprotein Profiles in Anti-Phosphotyrosine Kinase Assays.

The ability of the different mAbs to activate kinases was tested first by anti-PY immune complex kinase assays. In these studies, cells were activated for 5 min with multivalent antigen or with mAbs, then lysed and anti-phosphotyrosine antibodies used to precipitate tyrosine phosphorylated proteins as well as proteins co-precipitating with anti-phosphotyrosine-reactive species. The resulting immune complexes were incubated with [³²P]-ATP and phosphoproteins were separated by SDS-PAGE and detected by autoradiography. Typical results are illustrated in FIG. 2. All three mAbs induce more Lyn phosphorylation than antigen. Furthermore, Lyn phosphorylation was consistently greater in H10-treated cells than in cells activated with mAbs F4 and J17. These data suggest that H10-receptor dimers are fully competent to perform the initial activation of Lyn that launches the FcεR1 signalling sequence. Remarkably, there was strikingly more radiolabelled β and γ subunit when anti-PY immune complexes were generated from H10-activated cells in comparison with cells activated with antigen or the signalling-competent mAbs. These results could occur if anti-phosphotyrosine precipitated larger amounts of receptor subunits from H10-treated cells or if these subunits could be phosphorylated in vitro to higher specific activities than subunits from cells activated with antigen, F4 or J17.

### Example III

Different mAbs Induce Different Phospho-β Isoforms in RBL-2H3 Cells.

The amounts of FcεR1 β subunits in anti-phosphotyrosine immunoprecipitates from variously activated cells were examined by SDS-PAGE followed by immunoblotting with anti-β subunit antibodies (FIG. 3, panel A). Anti-phosphotyrosine immune complexes from resting cells (lane 1) were essentially free of β subunits, indicating there is little intrinsic subunit phosphorylation. Anti-phosphotyrosine complexes from cells activated for 5 min with antigen or with mAbs contained β subunits that varied not only in mount but in electrophoretic mobility. Specifically, three distinct β subunit isoforms were detectable by immunoblotting with anti-β Mab. The highest mobility isoform, designated β1, was detected exclusively in H10-treated cells. The intermediate mobility isoform, β2, was present in largest amount in H10-treated cells, but was also readily detected in immune complexes from cells treated with antigen and with mAbs F4 and J17. The slowest moving isoform, β3, was completely absent from H10-treated cells and was most abundant in antigen-treated cells. PhosphorImager measurements of the summed signal intensities from all three bands showed that anti-phosphotyrosine antibody immunoprecipitates at least twice as much total β subunit from H10-treated cells than from cells activated by antigen or the other mabs.

All three β subunit isoforms, β1 to β3, were detected by anti-phosphotyrosine blotting of anti-phosphotyrosine immune complexes (FIG. 2, panel B). However, the distribution of signal intensity between isoforms was different when detection was with anti-PY as compared to anti-β mAbs. We assumed that this differences reflected different levels of phosphorylation of the three isoforms, and explored this hypothesis by PhosphorImager analysis of the signals generated when replicate anti-PY gels are probed with anti-PY vs anti-β mAbs. For the gels in FIGS. 6A,B, the average ratios of PY signal intensity to β signal intensity were as follows: 0.15 for β1; 0.22 for β2; and 0.96 for β3. These numbers establish that the three isoforms reflect a hierarchy of β subunit phosphorylation, with the rapidly moving species, β1 and β2, found only in H10-treated cells representing the least phosphorylated form and the slow moving form, β3, that dominates in antigen-treated cells representing the most phosphorylated species.

### Example IV

Lyn does not Associate with the FcεR1 β3 Isoform.

In panels A and B of FIG. 4, anti-Lyn immune complexes were precipitated from lysates of antigen or Mab-activated cells, separated by SDS-PAGE and Western blots of replicate gels were probed as above, with either anti-β (4A) or anti-phosphotyrosine mAbs (4B). The two less phosphorylated β subunit isoforms, β1 and β2, discovered in highest amounts anti-phosphotyrosine immune complexes from H10-activated cells, are also prominent in anti-Lyn immune complexes and are thus identified as Lyn-binding isoforms. In contrast, the highly phosphorylated β3 isoform, found particularly in anti-PY immune complexes from antigen-treated cells, is completely absent from anti-Lyn immune complexes and is thus identified as having no binding activity for Lyn. These results indicate that the majority of Lyn in antigen-treated cells is free of associated receptor, whereas a substantial amount of Lyn exists in a stable complex with FcεR1 subunits in H10-treated cells.

The results of anti-Lyn immune complex kinase assays support this conclusion. In FIG. 4, anti-Lyn immune complexes from lysates of activated cells were incubated with [³²P]-ATP and phosphoproteins separated by SDS-PAGE. These data confirm that the majority of Lyn in antigen-treated cells is free of associated receptor subunits. There was also very little co-precipitating receptor subunit in Lyn immune complexes generated from cells activated with mAbs F4 or J17. In contrast, substantial amounts of β and γ subunits co-precipitated with Lyn from H10-activated cells and were phosphorylated in vitro by this kinase. Although the multiple phospho-β isoforms were sometimes resolved in in vitro kinase assays, this gel shows the more typical result in which phospho-β runs as a single band with the mobility of the most phosphorylated isoform. This hyperphosphorylation presumably reflects the release of restraints to complete phosphorylation under the conditions of our in vitro assays.

### Example V

H10-receptor complexes activate Lyn and support βγ subunit phosphorylation:

In FIG. 5A, anti-Lyn immune complexes from lysates of resting or activated cells were incubated with [³²P]-ATP and phosphoproteins separated by SDS-PAGE. As previously reported (J. Biol. Chem. 270: 4013-4022, 1995), Lyn immunoprecipitated from both resting and antigen-activated cells shows a modest level of in vitro_phosphorylation. Lyn immunoprecipitated from Mab-treated cells showed a stronger in vitro phosphorylation signal than antigen in this experiment. This could occur because Mab-receptor dimers are more effective than antigen at inducing Lyn activation. However, a more likely explanation is that the 5 min of crosslinking used in this study is optimal for Mab-induced Lyn activation but past the peak of antigen-induced Lyn activation (1-2 min). These kinase-specific assays confirm that all three mAbs can activate Lyn.

There was very little phosphorylated receptor subunit in anti-Lyn immune complex kinase reaction mixtures generated from cells activated with antigen or with mAbs F4 or J17. In contrast, substantial amounts of phosphorylated FcεR1 β and γ subunits were present in anti-Lyn immune complex kinase reaction mixtures from H10-treated cells. These results provided the first evidence that FcεR1 crosslinking with Mab H10 induces the formation of stable complexes between Lyn and its principal endogenous substrates, the receptor's β and γ subunits. This Lyn-bound receptor is a likely source of some or all of the excess phospho-β detected by anti-phosphotyrosine immune complex kinase assays in FIG. 4B.

### Example VI

H10-FcεR1 Dimers are Poor Syk Activators.

In FIG. 5B, anti-Syk immune complexes were incubated with [³²P]-ATP to measure crosslinked-induced Syk activation. Syk is strongly activated by antigen and Mab J17. There is an intermediate level of Syk activation in F4-treated cells. H10-receptor complexes induce very little Syk activation.

### Results

Contemporary models have suggested that the initial event in the FcεR1 signalling cascade is Lyn activation, resulting in FcεR1 β and γ subunit phosphorylation required for the recruitment and activation of Syk. The results of the above anti-phosphotyrosine immune complex kinase assays revealed more, not less, Lyn phosphorylation in H10-activated cells, indicating that H10-receptor dimers are fully competent to perform the initial activation of Lyn that launches the FcεR1 signalling sequence. The results of anti-Lyn immune complex kinase assays confirmed this result. There was strikingly more radiolabelled β and γ subunit when anti-phosphotyrosine or anti-Lyn immune complexes were generated from H10-activated cells in comparison with cells activated with antigen or the signalling-competent mAbs. Nevertheless, there was less Syk activation in H10-activated cells than in cells activated with antigen or with mAbs F4 and J17. This indicated the poor signalling activity of H10-receptor dimers on events between Lyn-mediated receptor subunit phosphorylation and Syk activation.

The results of SDS-PAGE separation of anti-phosphotyrosine immune complexes followed by anti-β and anti-phosphotyrosine immunoblotting confirmed that more FcεR1 β subunit immunoprecipitates with anti-phosphotyrosine from H10-activated cells than from cells activated with antigen or other mAbs. Importantly, these studies also established that the FcεR1 β subunit exists in at least three distinct phosphorylation states, each with its own characteristic electrophoretic mobility, in activated cells. Results obtained by SDS-PAGE separation of anti-Lyn immune complexes followed by anti-β and anti-phosphotyrosine immunoblotting showed that only the two less phosphorylated β subunit isoforms most characteristic of H10-treated cells associate with Lyn. In contrast, the highly phosphorylated β3 isoform most characteristic of antigen-treated cells has no binding activity towards Lyn.

What is claimed is:

1. An antagonist for curtailing signal propagation in an immune cell along a MIRR-coupled signalling pathway characterized by Src-PTK-mediated multichain immune recognition receptor (MIRR) phosphorylation accompanied by the formation of unstable Src/MIRR complexes and followed by Syk PTK activation, comprising an Src sequestering agent capable of stabilizing the complexes against dissociation.

2. The antagonist of claim 1, wherein the sequestering agent is a signal-curtailing ligand also capable of initiating Src-PTK-mediated MIRR phosphorylation.

3. The antagonist of claim 1, wherein the ligand cross-links extracellular domains of the MIRR.

4. The antagonist of claim 3, wherein the ligand cross-links the domains into dimers.

5. The antagonist of claim 3, wherein the ligand is a natural antigen for the receptor or an anti-receptor antibody.

6. The antagonist of claim 5, wherein the ligand is a monoclonal antibody specific for the MIRR extracellular domains.

7. A pharmaceutical composition comprising at least one antagonist of claims 1-5 or 6 and a pharmaceutically-acceptable carrier.

8. The composition of claim 7 which is a vaccine.

9. A method for inhibiting one or more immune cell effector functions, comprising curtailing signal propagation in the cell along at least one MIRR-coupled signalling pathway characterized by Src-PTK-mediated MIRR phosphorylation accompanied by the formation of unstable Src/MIRR complexes and followed by Syk-PTK activation, to inhibit Syk-PTK activation.

10. The method of claim 9, wherein signal propagation is curtailed by sequestering Src in a Src/MIRR complex, with an Src sequestering agent.

11. A method for curtailing signal propagation in an immune cell along a MIRR-coupled signalling pathway normally characterized by Src-PTK mediated MIRR phosphorylation accompanied by the formation of unstable Src/MIRR complexes and followed by Syk PTK activation, comprising contacting the cell with a Src sequestering agent which stabilizes the complexes and inhibits Syk PTK activation

12. The method of claim 10 or 11, wherein the Src sequestering agent additionally initiates propagation of the signal by cross-linking extracellular domains of the MIRR.

13. The method of claim 10 or 11, wherein the Src sequestering agent is a monoclonal antibody specific for the MIRR extracellular domains.

14. A method for inhibiting immune cell activation in a mammal, comprising treating the animal with an antagonist according to claims 1-5 or 6.

15. A method for inhibiting immune cell activation in a mammal, comprising treating the animal with a pharmaceutical composition comprising at least one antagonist of claims 1-5 or 6 and a pharmaceutically-acceptable carrier.
